(19) United States
(12) Patent Application Publication
(10) Pub. No.: US 2011/0237980 A1
(43) Pub. Date: Sep. 29, 2011

(54) Assessment and classification system

(75) Inventors: Christopher L. Owens, Phoenix, AZ (US); Jeanne K. Pomatto-Hertz, Scottsdale, AZ (US); Timothy R. Littlefield, Phoenix, AZ (US)

(73) Assignee: CRANIAL TECHNOLOGIES, INC.

(21) Appl. No.: 12/798,076

(22) Filed: Mar. 29, 2010

(51) Int. Cl. A61B 5/103

(52) U.S. Cl. 600/587

(57) ABSTRACT

The present invention provides assessment of cranial deformities. One embodiment for providing an on-line assessment comprises providing a server; and providing a database accessible by the server. The database comprises a plurality of image sets. Each image set comprises a plurality of images of a corresponding head shape selected from a plurality of head shapes. Each image is an image of the corresponding head shape from one viewing orientation of a plurality of predetermined viewing orientations. In one embodiment, the plurality of head shapes comprises a normal head shape and one or more head shapes selected from the group comprising a plagiocephalic head shape, a brachycephalic head shape, and a scaphocephalic head shape.

### FIELD OF THE INVENTION

The present invention pertains to the classifying a three-dimensional object, in general, and to classifying a head shape to assess the type and severity of cranial deformity, in particular.

### BACKGROUND OF THE INVENTION

While there is no such thing as a “perfect head” there is definitely a “norm” for infant heads. Typically, the average skull is one third longer than it is wide, and is typically, although not perfectly, symmetric.

A common cranial deformity is plagiocephaly. Plagiocephaly literally means “oblique head” (from Greek: plagio=oblique, and cephale=head). It is also called a parallelogram deformity because when viewing down on the infant's head, the head forms a parallelogram shape. From this angle, it looks as if half of the head has been pushed forward. The head deformity is often accompanied by misalignment of the ears, facial asymmetry, and bulging forehead.

Another common cranial deformity is brachycephaly. With brachycephaly the head is flattened uniformly across the back, causing a wider and shorter head shape. Increased head height is also common in children with brachycephaly

An additional common cranial deformity is scaphocephaly. Scaphocephaly, also known as dilichocephaly, describes a head with a long, narrow shape. It is particularly common in premature babies who spend time in neonatal intensive care units NICU, or from a breech intrauterine orientation.

A further common cranial deformity is brachycephaly with plagiocephaly This deformity occurs when there is a combination of brachycephaly and plagiocephaly present.

All the above-described cranial deformities have become more prevalent since the “Back to Sleep” Campaign was initiated to help reduce the occurrence of sudden infant death syndrome (“SIDS”).

### SUMMARY

In accordance with the principles of the invention, a method and apparatus are provided that are utilized to classify the type of a particular complex object by comparing images of a subject object with images representative of one or more representative predetermined types of objects, utilizing the type of complex object identified by the comparison to select a group of second images against which the images of the subject object are compared to determine a second characteristic of the subject object. In accordance with an aspect of the invention, the images of the subject object are image views in predetermined viewing orientations, and second images representative of the second characteristics are likewise image views in the same predetermined viewing orientations.

In accordance with the principles of the invention, a method and apparatus are provided that are utilized to determine the type of a particular medical condition by comparing images of a subject with images representative of one or more medical conditions, utilizing the type of medical condition identified to select a group of second images against which the images of the subject are compared to determine the severity of the medical condition. In accordance with an aspect of the invention, the subject images are provided in predetermined viewing orientations, and the images representative of one or more medical conditions are likewise image views provided in the same predetermined viewing orientations.

The illustrative embodiment provides methods and apparatus to determine the type of an infant's head shape, i.e., plagiocephaly, brachycephaly, scaphocephaly, and brachycephaly with asymmetry, by comparing the subject infant's head shape to images of other infant's head shapes, where the other images point out different characteristics associated with each type of deformity.

In the illustrative embodiment the severity of the deformity of the subject infant's head shape, scaled from normal to severe, is determined by comparing the subject infant's head shape to second images of other infant's head shapes, where the second images point out the progression of the distortion of head shapes as the type of deformity progresses from mild to severe.

The present invention provides a method and apparatus for the classifying a head shape by comparative assessment of a head shape against images of predetermined types of head shapes such that the type and severity of cranial deformities may be determined.

A method for providing assessment of cranial deformation of a subject's head, comprises providing a server and providing a database accessible by the server. The database comprises a first plurality of first image sets utilizable to assess a cranial deformity type and a second plurality of second image sets utilizable to assess the severity of a cranial deformity. The method includes utilizing the database to access the first plurality of first image sets to assess a type of cranial deformity of the subject's head; and utilizing the database to access the second plurality of second image sets to assess the severity of the cranial deformation of the subject's head.

An embodiment of the method further comprises providing a set of third images of the subject's head and comparing the third images with the plurality of first image sets to assess a type of cranial deformity.

In accordance with the embodiment, the method includes selecting one of the second image sets corresponding to the assessed type of cranial deformity; and comparing the third images with the selected one of said second image sets to assess the severity of the assessed type of cranial deformity.

In a particularly advantageous embodiment the method comprises automatically comparing the third images with the first plurality of first image sets to assess a type of cranial deformity; automatically selecting one of the second image sets corresponding to the assessed type of cranial deformity; and automatically comparing the third images with the selected one of the second image sets to assess the severity of the assessed type of cranial deformity.

In the embodiment, the method comprises selecting each first image set to comprise a group of images including one image for each of a plurality of head shapes. The plurality of head shapes comprises a normal head shape and one or more head shapes selected from the group comprising a plagiocephalic head shape, a brachycephalic head shape, and a scaphocephalic head shape.

Still further in accordance with the embodiment each first set of images is selected such that all head shape images are views of the corresponding head from one viewing orientation of a plurality of predetermined viewing orientations.

The predetermined viewing predetermined viewing orientations of said plurality of viewing orientations from: a first predetermined viewing orientation looking down at a head from above, a second viewing orientation from the side looking at the side of a head, a third viewing orientation from the front looking at the front of a head, and a fourth viewing orientation from below looking up at head towards the jaw of the head.

In another embodiment of the invention, the method comprises providing a downloadable application program and providing the database accessible by the application program.

In another embodiment of the invention, a combination is provided, the combination comprises an application program operable to provide assessment of cranial deformities and a database accessible by the application program. The database comprises a first plurality of first image sets utilizable to assess a cranial deformity type and a second plurality of second image sets utilizable to assess the severity of a cranial deformity. The application program utilizes the database to access the first plurality of first image sets to assess a type of cranial deformity of a subject's head. The application utilizes the database to access the second plurality of second image sets to assess the severity of the cranial deformation of the subject's head.

Apparatus operable to provide assessment of cranial deformities comprises programmable apparatus comprising a display and an application program including a database accessible by the application program. The database comprises a plurality of image sets, each set comprising a plurality of images of a corresponding head shape selected from a plurality of head shapes. Each image is an image of the corresponding head from one viewing orientation of a plurality of predetermined viewing orientations. The application program is operable to display selected groups of images such that a user of the apparatus may compare the selected group of images with the shape of a subject's head viewed from the same one predetermined viewing orientation.

### BRIEF DESCRIPTION OF THE DRAWING FIGURES

The invention will be better understood from a reading of the following detailed description in conjunction with the drawing figures in which like reference designators identify like elements, and in which:

FIG. 1 illustrates a system in accordance with the principles of the invention;

FIG. 2 shows a first set of images stored in the database of FIG. 1;

FIG. 3 shows a second set of images stored in the database of FIG. 1;

FIG. 4 shows a third set of images stored in the database of FIG. 1;

FIG. 5 shows a fourth set of images stored in the database of FIG. 1;

FIG. 6 shows an image stored in the database of FIG. 1;

FIG. 7 shows fifth, sixth and seventh sets of images stored in the database of FIG. 1; and

FIG. 8 illustrates a second system in accordance with the principles of the invention.

### DETAILED DESCRIPTION

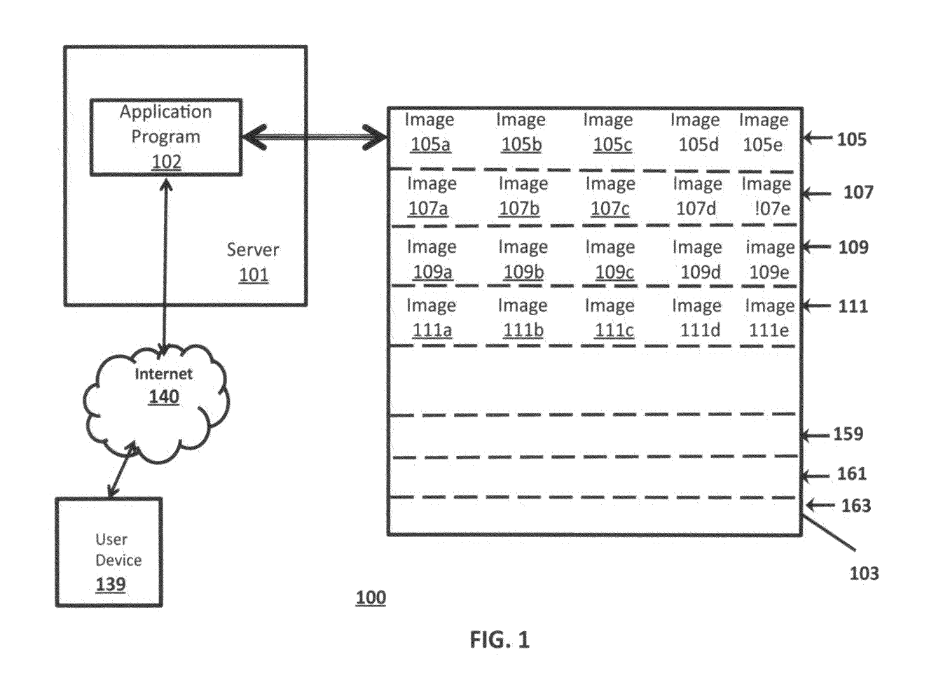

Turning to FIG. 1, system 100 of the invention includes a server 101 and a database 103. Database 103 comprises a plurality of first image sets 105, 107, 109, 111 of images. Each first image set 105, 107, 109, 111 comprises a plurality of images of a different predetermined head shape. The images in each first image set are images of the corresponding head shapes viewed from a different orientation selected from a set of predetermined viewing orientations.

In the embodiment shown in FIG. 1, first image set 105 is a set of images 105a, 105b, 105c, 105d of a normally shaped head, a plagiocephalic head, a brachycephalic head shape and a scaphocephalic head shape all viewed in a first predetermined viewing orientation.

First image set 107 is a set of images 107a, 107b, 107c, 107d of a normally shaped head, a plagiocephalic head, a brachycephalic head shape and a scaphocephalic head shape all viewed in a second predetermined viewing orientation.

First image set 109 is a set of images 109a, 109b, 109c, 109d of a normally shaped head, a plagiocephalic head, a brachycephalic head shape and a scaphocephalic head shape all viewed in a third predetermined viewing orientation.

First mage set 111 is a set of images 111a, 111b, 111c, 111d of a normally shaped head, a plagiocephalic head, a brachycephalic head shape and a scaphocephalic head shape all viewed in a fourth predetermined position.

The plurality of predetermined viewing orientations of first image sets 105, 107, 109, 111 are selected from a first predetermined viewing orientation looking down at a head from above, a second viewing orientation from the side looking at the side of a head, a third viewing orientation from the front looking at the front of a head, and a fourth viewing orientation from below looking up at head towards the jaw of the head.

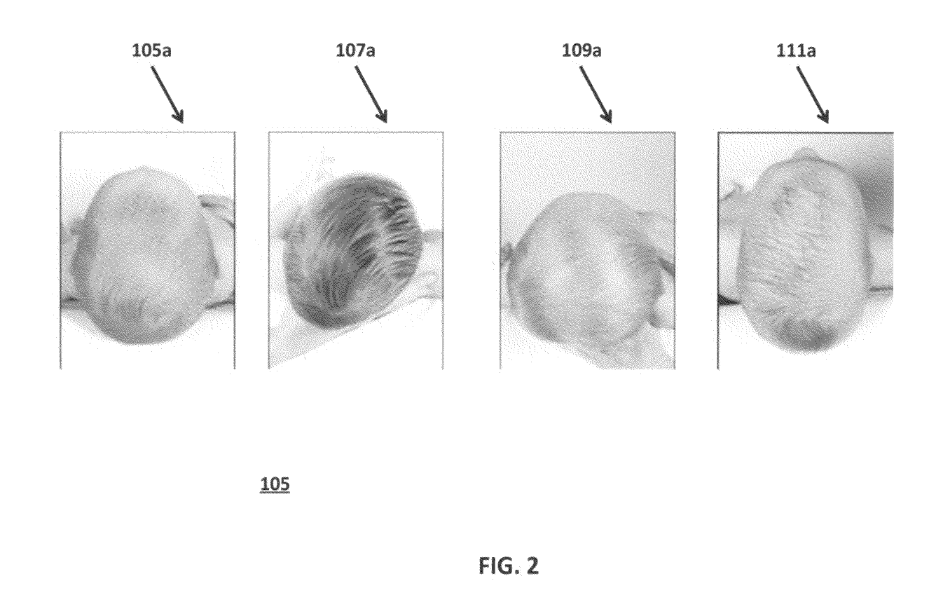

Turning to FIG. 2, first image set 105 is shown as comprising a set of images all from the first viewing orientation of viewing the head shapes from above. FIG. 105a is an image of a normal head viewed from above. FIG. 105b is a view of a plagiocephalic head viewed from above. FIG. 105c is a view of a brachycephalic head shape viewed from above. FIG. 105d is a view of a scaphocephalic head shape viewed from above.

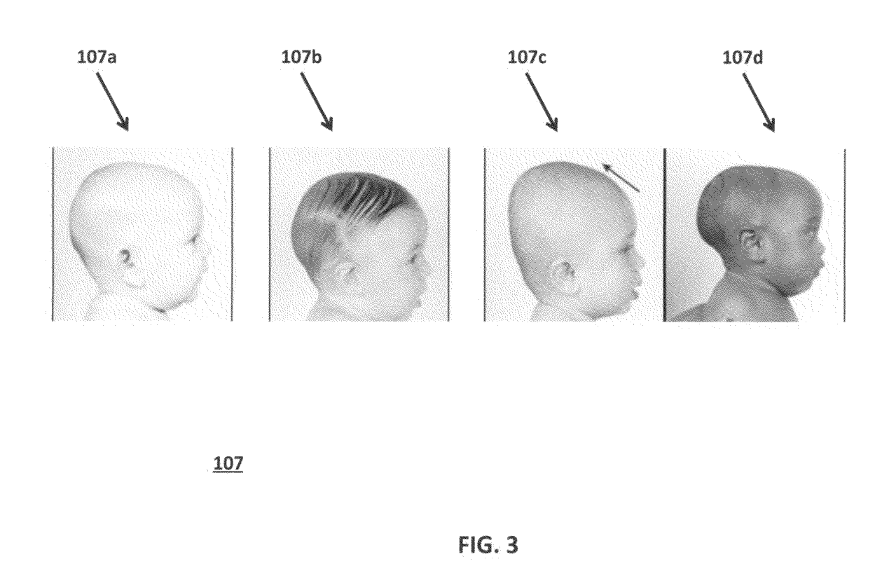

Turning to FIG. 3, first image set 107 is shown as comprising a set of images all from the second viewing orientation of viewing the head shapes from one side. FIG. 107a is an image of a normal head viewed from the side. FIG. 107b is a view of a plagiocephalic head viewed from the side. FIG. 107c is a view of a brachycephalic head shape viewed from the side. FIG. 107d is a view of a scaphocephalic head shape viewed from the side.

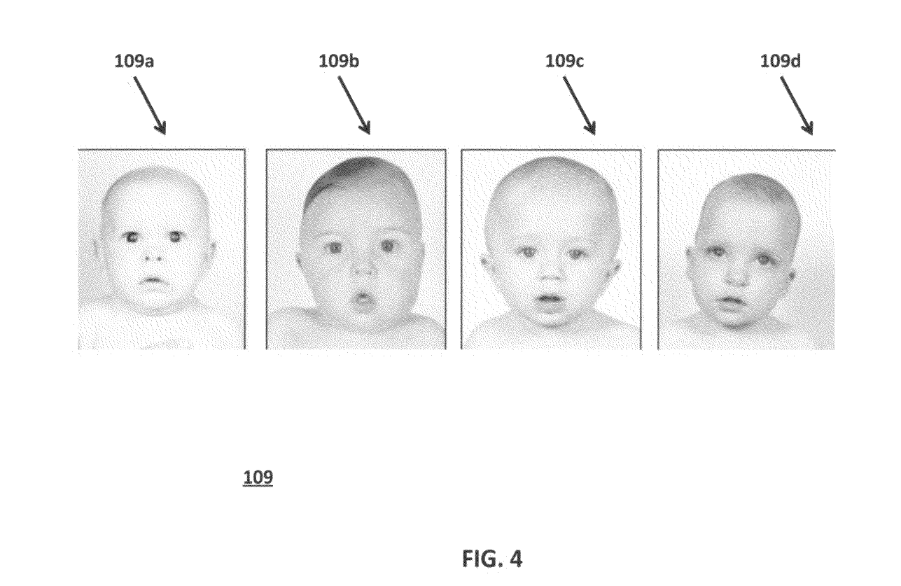

Turning to FIG. 4, first image set 109 is shown as comprising a set of images all from the third viewing orientation of viewing the head shapes from in front. FIG. 109a is an image of a normal head viewed from the front. FIG. 109b is a view of a plagiocephalic head viewed from the front. FIG. 109c is a view of a brachycephalic head shape viewed from the front. FIG. 109d is a view of a scaphocephalic head shape viewed from the front.

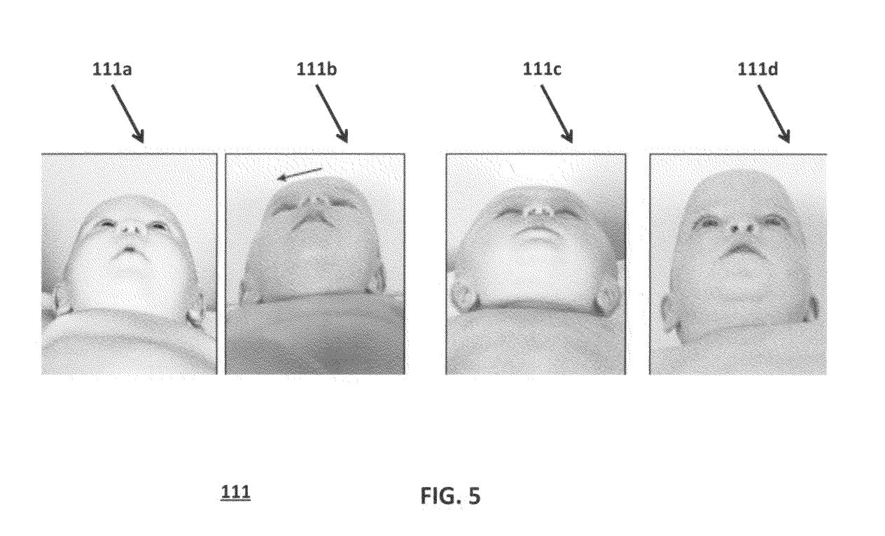

Turning to FIG. 5, first image set 111 is shown as comprising a set of images all from the first viewing orientation of viewing the head shapes from below. FIG. 111a is an image of a normal head viewed from below. FIG. 111b is a view of a plagiocephalic head viewed from below. FIG. 111c is a view of a brachycephalic head shape viewed from below. FIG. 111d is a view of a scaphocephalic head shape viewed from below.

Turning back to FIG. 1, server 101 comprises an application program APP that is utilized to retrieve first image sets 105, 107, 109, 111 from database 103. Server 101 is accessible via the Internet 141. A remote user at an apparatus or device 139 that has access to the Internet 141 may be used to request that server 101 download image groups 105, 107, 109, 111. Device 139 may be any one of a number of devices that can access the Internet and includes computers, cellular phones, and any number of wired and wireless devices. Typically device 139 will include a display portion that can display the downloaded images from server 101.

In typical use or operation, a user at device 139 performs any one of a number of conventional operations that are commonly used to perform an Internet download to cause server 101 to download a first image set 105 which includes a views of the head shapes from above. The user would typically view a subject's head in the same viewing orientation and compare the subject's head shape to each of the images 105a, 105b, 105c, 105d in the first image set 105 and note which shape most closely corresponds to that of the subject's head shape.

Although not shown in the drawing Figures, textual descriptions are stored along with the image shapes. Stored in conjunction with said first image 105a is a textual description of the shape of a normal head. The textual description is that: “Back of the head is curved. Both sides of the head match.” Stored in conjunction with second image 105b is a textual description of the plagiocephalic head viewed from the first position that states: “One ear is closer to the shoulder on one side of the head. One side of the head looks higher than the other.” Stored in conjunction with third image 105c is a textual description of the brachycephalic head viewed from the first position that states: “The head is shorter length front to back. The head is of tall height. The forehead slopes back. The back of the head is flat.” Stored in conjunction with fourth image 105d is a textual description of the scaphocephalic head viewed from the first position that states: “The head is greater in length front to back.”

Next, the user at device 139 downloads from server 101 first image set 107 shown in FIG. 3 which includes a views of the head shapes. The user would typically view the subject's head in the same viewing orientation and compare the subject's head shape to each of the images 107a, 107b, 107c, 107d.

Stored in conjunction with image 107a is a textual description of the shape of a normal head that states: “Back of the head is curved. Both sides of the head match.” Stored in conjunction with image 107b is a textual description of the plagiocephalic head that states: One ear is closer to the shoulder on one side of the head. One side of the head looks higher than the other.” Stored in conjunction with image 107c is a textual description of the brachycephalic head that states: “The head is shorter length front to back. The head is of tall height. The forehead slopes back. The back of the head is flat.” Stored in conjunction with image 107d is a textual description of a scaphocephalic head that states: “The head is greater in length front to back.”

Next, the user at device 139 downloads from server 101a third image set 109 shown in FIG. 4 which includes a views of the head shapes viewed from a third position disposed directly in front of the heads. The user would typically view the infant's head in the same viewing orientation and compare the infant's head shape to each of the images 109a, 109b, 109c, 109d in image group 109.

Stored in association with image 109a is a textual description of the shape of a normal head that states: “Eyes are the same size and at the same level on the face. Cheeks are the same size. Top of head is level.” Stored in association with image 109b is a textual description of a plagiocephalic head that states: “One eye is smaller than the other. One cheek is fuller than the other. Top of head is not level.” Stored in conjunction with third image 109c is a textual description of the brachycephalic head that states: “Face appears small to the size of the head. Head is wide. Bump above the ears creates the widest part of the head. Ears poke out at top.” Stored in conjunction with fourth image 109d is a textual description of the scaphocephalic head that states: “Increased head height. Head is narrow.”

In addition, a general textual description is provided with first image set 109 that is downloaded to user device 139. That description states: “When looking at your baby's face; the best way to see any asymmetry is to place the baby in front of a mirror and look at their reflection. Because you are used to seeing your baby head on, this view in the mirror will allow you to see any differences.”

Next, the user at device 139 downloads from server 101a first image set 111 shown in FIG. 5 which includes a views of the head shapes viewed from a fourth position disposed below the heads. The user would typically view the infant's head in the same viewing orientation and compare the infant's head shape to each of the images 111a, 111b, 111c, 111d in image group 111.

In the fourth viewing position, the image subjects are lying on their backs with their nose pointing toward the ceiling and the image is from below looking up at head towards the jaw of the head.

A first image 111a is the shape of a normal head. Stored in conjunction with said first image 111a is a textual description of the shape of a normal head. The textual description is that: “Eyes and ears are the same on both sides. Forehead is arched and level.” Stored in conjunction with second image 111b is a textual description of the plagiocephalic head as viewed from the fourth position that states: “Forehead is angled and not level. Face has a slope and seems to be lopsided. Eyes and ears are not level. One cheek and side of the face is more full than the other.” Stored in conjunction with third image 111c is a textual description of the brachycephalic head that states: “Head is wide. Bump above ears is the widest point of the head.” Stored in conjunction with fourth image 111d is a textual description of the scaphocephalic head that states: “Head is narrow. Forehead is square.”

By viewing the downloaded images, an assessment may be made with respect to the type of cranial deformity.

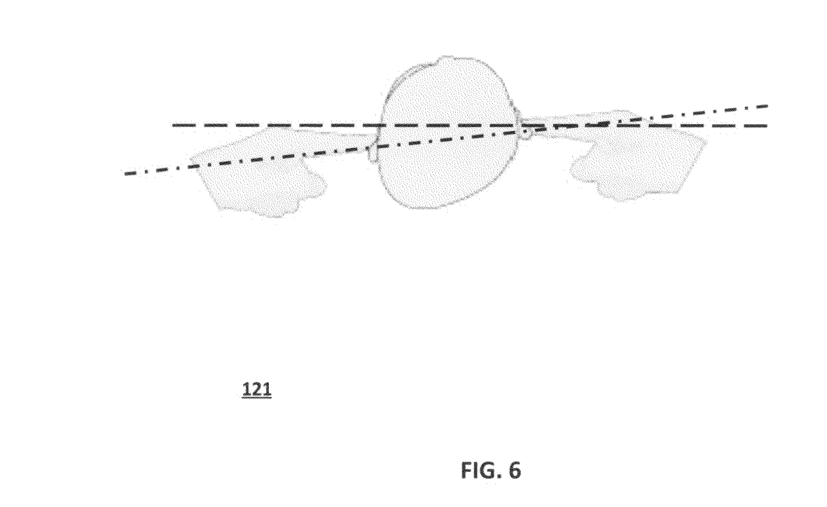

In the event the assessment is that the infant has a plagiocephaly head shape, a further image or image set 121 may be downloaded from database 103. Image group 121 is a view looking down at the top of an infant's head. As shown in FIG. 6, an image 121 shows an index finger in each ear. The downloaded text states: “Now let's look at your baby's ears. Please put an index finger in each of the baby's ears while they are still sitting on someone's lap. Then stand over the baby and look to see if your fingers line up. The greater difference in the ear position helps tell us how far along your child's plagiocephaly has progressed.”

Although image set 121 shown in FIG. 6 includes only one image 107e, it will be apparent to those skilled in the art that a plurality of images may be provided, each showing images of heads with different degrees of plagiocephaly.

System 100 may be further used to provide an assessment of the severity of the deformation of the subject's head. Second image sets 159, 161, 163 are stored in database 103 that are used to provide comparative determination of severity of deformation. Second image set 159 comprises a set of images of plagiocephalic head shapes of different severity. Second image set 161 comprises a set of images of brachycephalic head shapes of different severity. Second image set 163 comprises a set of images of scaphocephalic head shapes of different severity.

The user at device 139, after making an assessment of the type of cranial deformity utilizes that assessment to download a corresponding one of the second image sets 159, 161, 163 to assess the severity of the deformity.

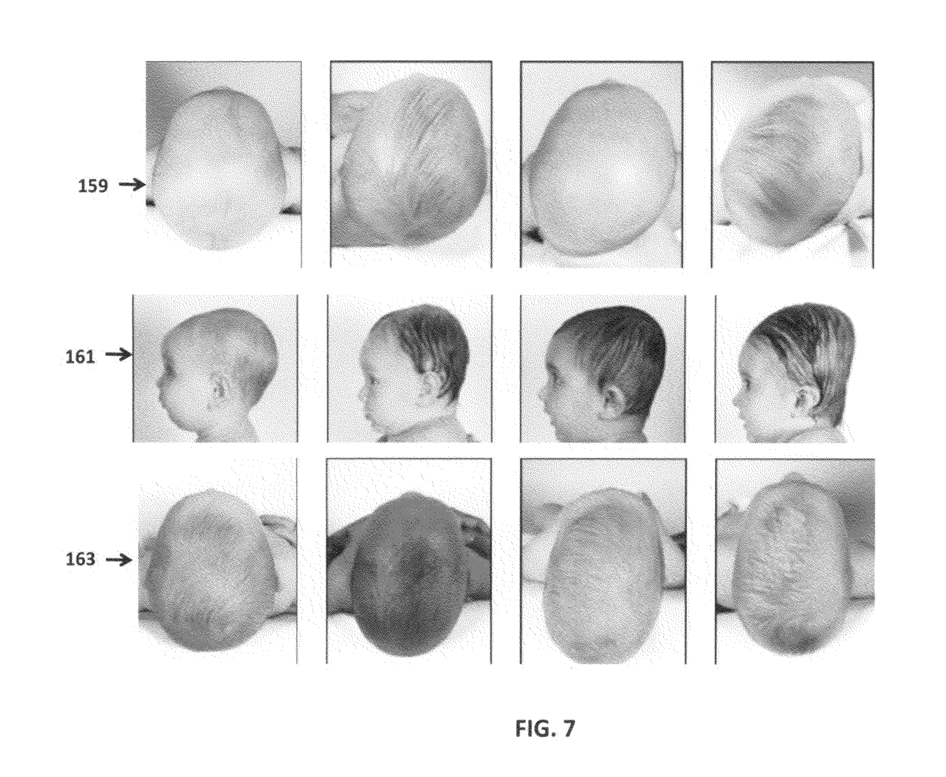

Turning now to FIG. 7, second image sets 159, 161, 163 are shown. Second image set 159 comprises a view of a normal head shape and plagiocephalic head shapes at different levels of severity. To the right of the normal head shape, the images respectively represent mild, moderate and severe plagiocephalic head shapes. The user at device 139 by comparing the subject's head with the shapes shown in second image set 159 may make a standardized assessment of the severity of the deformity.

Second image set 161 comprises a view of a normal head shape and brachycephalic head shapes at different levels of severity. To the right of the normal head shape, the images respectively represent mild, moderate and severe brachycephalic head shapes. The user at device 139 by comparing the subject's head with the shapes shown in second image set 161 may make a standardized assessment of the severity of the deformity.

Second image set 163 comprises a view of a normal head shape and scaphocephalic head shapes at different levels of severity. To the right of the normal head shape, the images respectively represent mild, moderate and severe scaphocephalic head shapes. The user at device 139 by comparing the subject's head with the shapes shown in second image set 163 may make a standardized assessment of the severity of the deformity.

In a second embodiment of the invention, images of a subject's head may be uploaded or otherwise provided to server 101. Application program 102 may further be operable to compare the uploaded images of the subject's head to the image sets stored in database 103 to both automatically determine the nature of the deformity and then automatically determine the severity of the deformity in accordance with the standards represented by the stored images. It will be appreciated by those skilled in the art that various algorithms may be utilized to perform the assessment of deformity type and severity of deformity. In other embodiments, the uploaded images of the subject's head may be directly compared to image sets that are used to automatically determine both the type and severity of assessments at the same time.

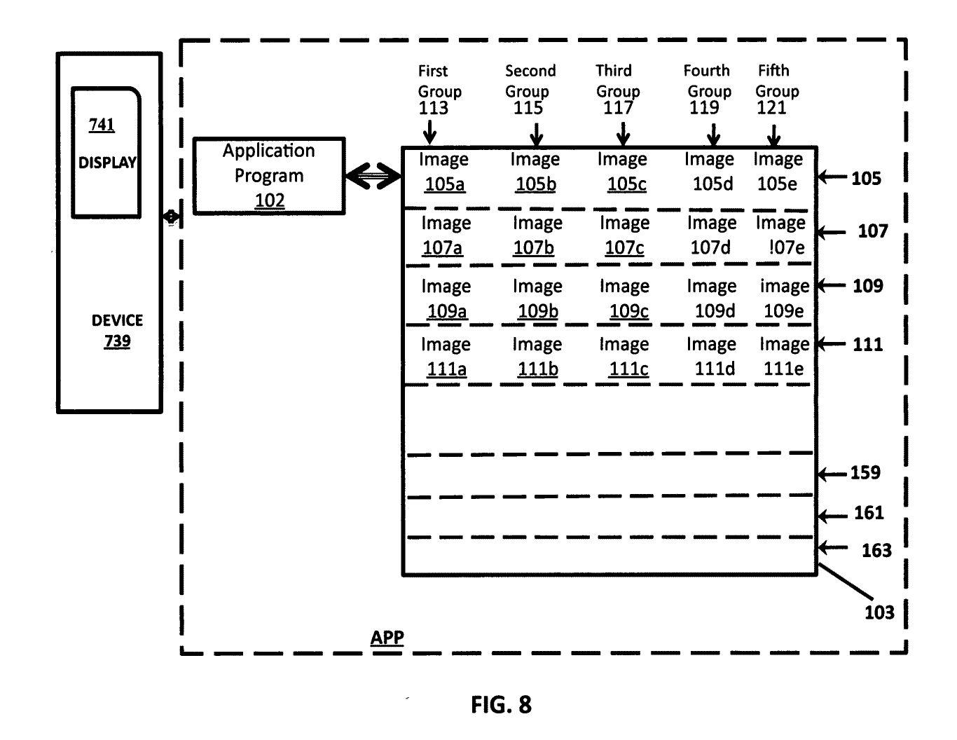

Turning now to FIG. 8, in another embodiment, application APP for providing assessment of cranial deformities is downloadable to device 739 and is executable directly on device 739. Application APP includes application program 102 and database 103 accessible by application program 102.

Device 739 may be a remote apparatus comprising a display 741. The application program is operable on the remote apparatus device 739 to display the selected first sets 105, 107, 109, 111 and second image sets 159, 161, 163 such that a user of said apparatus may compare the selected image sets with the shape of an infant's head viewed from the same one predetermined viewing orientation. Device 739 may be any of a large number of apparatus including various, computers, Internet devices, data terminals, text and display apparatus, wireless communication devices such as cellular telephones, or any other device in which a memory, a display, and an inputting apparatus are provided.

The operation of device 739 with downloadable application APP is the same as the operation described above with respect to FIG. 1. In other words the user of device 739 may access various first image sets 105, 107, 109, 111 and second image sets 159, 161, 163 from database 103 for display on display 741 to determine the type of deformity and also the severity of the deformity.

In certain embodiments, device 739 may include a camera which is utilized to capture images of a subject's head that application program 102 automatically compares to the images stored in database 103 to automatically determine the type of deformity and the severity of the deformity.

In some embodiments of the invention application APP is not downloaded from the Internet or a server, but rather is provided on a memory device including, but not limited to memory devices such as compact disk, DVD, flash memory or similar and equivalent memory devices.

It will be particularly appreciated by those skilled in the art that the present invention is not limited to assessment of heads, but may equally be utilized to assess the type and severity of various medical conditions that are ascertainable from an image of the condition. Even further, the present invention may be utilized to categorize various three-dimensional objects according to type and to further provide a classification or assessment within the type.

It will be appreciate by those skilled in the art that various changes and modifications may be made to the embodiments described herein without departing from the spirit or scope of the invention. It will be further appreciated that it is not intended to limit the invention to the embodiments shown and described herein and that the invention is intended to be limited in scope only by the claims appended hereto.

What is claimed is:

1. A method for classifying a three-dimensional object of complex shape, comprising:
providing a server;
providing a database accessible by said server,
said database comprising a first plurality of first image sets utilizable to assess a the shape of a three-dimensional object by type and a second plurality of second image sets utilizable to provide a further assessment of the object;
utilizing said database to access said first plurality of first image sets to assess a the type of said object; and
utilizing said database to access said second plurality of second image sets to assess characteristics of said object.

2. A method in accordance with claim 1, comprising:
utilizing an assessed type of said object to select a corresponding one said second image set of said second plurality of image sets.

3. A method in accordance with claim 1, comprising:
uploading a set of third images of said object;
comparing said third images with said first plurality of first image sets to assess a type of object.

4. A method in accordance with claim 3; comprising:
selecting one of said second image sets corresponding to said assessed type of object; and
comparing said third images with said selected one of said second image sets to assess characteristics of said object.

5. A method in accordance with claim 1, comprising
providing a set of third images of said object; and
automatically comparing said third images with said first plurality of first image sets to assess a type of said object.

6. A method in accordance with claim 5, comprising:
automatically selecting one of said second image sets corresponding to said assessed type of said object; and
automatically comparing said third images with said selected one of said second image sets to assess characteristics of said object.

7. A method for providing assessment of medical conditions, comprising:
providing a server;
providing a database accessible by said server,
said database comprising a first plurality of first image sets of a plurality of medical conditions utilizable to assess a type of medical conditions and a second plurality of second image sets utilizable to assess the severity of corresponding ones of said medical conditions;
utilizing said database to access said first plurality of first image sets to assess a type of medical condition; and
utilizing said database to access said second plurality of second image sets to assess the severity of the assessed medical condition.

8. A method in accordance with claim 7, comprising:
utilizing an assessed type of medical condition to select a corresponding one said second image set of said second plurality of image sets.

9. A method in accordance with claim 8, comprising:
providing a set of third images of a medical condition to be assessed;
comparing said third images with said first plurality of first image sets to assess a type of medical condition.

10. A method in accordance with claim 9, comprising:
selecting one of said second image sets corresponding to said assessed type of medical condition; and
comparing said third images with said selected one of said second image sets to assess the severity of said assessed type of medical condition.

11. A method in accordance with claim 7, comprising
providing a set of third images of a subject's medical condition; and
automatically comparing said third images with said first plurality of first image sets to assess a type of medical condition.

12. A method in accordance with claim 11, comprising:
automatically selecting one of said second image sets corresponding to said assessed type of medical condition; and
automatically comparing said third images with said selected one of said second image sets to assess the severity of said assessed type of medical condition.

13. A method for providing assessment of cranial deformation of a subject's head, comprising:
providing a server;
providing a database accessible by said server,
said database comprising a first plurality of first image sets utilizable to assess a cranial deformity type and a second plurality of second image sets utilizable to assess the severity of a cranial deformity;
utilizing said database to access said first plurality of first image sets to assess a type of cranial deformity of said subject's head; and
utilizing said database to access said second plurality of second image sets to assess the severity of said cranial deformation of said subject's head.

14. A method in accordance with claim 13, comprising:
utilizing an assessed type of cranial deformity to select a corresponding one said second image set of said second plurality of image sets.

15. A method in accordance with claim 13, comprising:
uploading a set of third images of said subject's head;
comparing said third images with said first plurality of first image sets to assess a type of cranial deformity.

16. A method in accordance with claim 15, comprising:
selecting one of said second image sets corresponding to said assessed type of cranial deformity; and
comparing said third images with said selected one of said second image sets to assess the severity of said assessed type of cranial deformity.

17. A method in accordance with claim 13, comprising
uploading a set of third images of said subject's head; and
automatically comparing said third images with said first plurality of first image sets to assess a type of cranial deformity.

18. A method in accordance with claim 17, comprising:
automatically selecting one of said second image sets corresponding to said assessed type of cranial deformity; and
automatically comparing said third images with said selected one of said second image sets to assess the severity of said assessed type of cranial deformity.

19. A method in accordance with claim 13, comprising:
selecting each said first image set to comprise a group of images including one image for each of a plurality of head shapes, said plurality of head shapes comprising a normal head shape and one or more head shapes selected from the group comprising a plagiocephalic head shape, a brachycephalic head shape, and a scaphocephalic head shape.

20. A method in accordance with claim 19, comprising:
selecting each said first set of images such that all of said first sets corresponding head shape images are a view of said corresponding head from one viewing orientation of a plurality of predetermined viewing orientations.

21. A method in accordance with claim 20, comprising:
selecting each predetermined viewing orientation of said plurality of viewing orientations from: a first predetermined viewing orientation looking down at a head from above, a second viewing orientation from the side looking at the side of a head, a third viewing orientation from the front looking at the front of a head, and a fourth viewing orientation from below looking up at head towards the jaw of the head.

22. A method in accordance with claim 21, comprising:
utilizing an assessed type of cranial deformity to select a corresponding one said second image set of said second plurality of image sets.

23. A method in accordance with claim 21, comprising:
providing a set of third images of said subject's head;
comparing said third images with said first plurality of first image sets to assess a type of cranial deformity.

24. A method in accordance with claim 23, comprising:
selecting one of said second image sets corresponding to said assessed type of cranial deformity; and
comparing said third images with said selected one of said second image sets to assess the severity of said assessed type of cranial deformity.

25. A method in accordance with claim 21, comprising
providing a set of third images of said subject's head; and
automatically comparing said third images with said first plurality of first image sets to assess a type of cranial deformity.

26. A method in accordance with claim 25, comprising:
automatically selecting one of said second image sets corresponding to said assessed type of cranial deformity; and
automatically comparing said third images with said selected one of said second image sets to assess the severity of said assessed type of cranial deformity.

27. A method for providing assessment of cranial deformities of a subject's head, comprising:
providing a downloadable application program;
providing a database accessible by said application program, said database comprising a first plurality of first image sets utilizable to assess a cranial deformity type and a second plurality of second image sets utilizable to assess the severity of a cranial deformity;
utilizing said database to access said first plurality of first image sets to assess a type of cranial deformity of said subject's head; and
utilizing said database to access said second plurality of second image sets to assess the severity of said cranial deformation of said subject's head.

28. A method in accordance with claim 27, comprising:
utilizing an assessed type of cranial deformity to select a corresponding one said second image set of said second plurality of image sets.

29. A method in accordance with claim 28, comprising:
providing a set of third images of said subject's head;
comparing said third images with said first plurality of first image sets to assess a type of cranial deformity.

30. A method in accordance with claim 29, comprising:
selecting one of said second image sets corresponding to said assessed type of cranial deformity; and
comparing said third images with said selected one of said second image sets to assess the severity of said assessed type of cranial deformity.

31. A method in accordance with claim 27, comprising
providing a set of third images of said subject's head; and
automatically comparing said third images with said first plurality of first image sets to assess a type of cranial deformity.

32. A method in accordance with claim 27, comprising:
providing a set of third images of said subject's head;
automatically selecting one of said second image sets corresponding to said assessed type of cranial deformity; and
automatically comparing said third images with said selected one of said second image sets to assess the severity of said assessed type of cranial deformity.

33. A method in accordance with claim 27, comprising:
selecting each said first image set to comprise a group of images including one image for each of a plurality of head shapes, said plurality of head shapes comprising a normal head shape and one or more head shapes selected from the group comprising a plagiocephalic head shape, a brachycephalic head shape, and a scaphocephalic head shape.

34. A method in accordance with claim 33, comprising:
selecting each said first set of images such that all of said first sets corresponding head shape images are a view of said corresponding head from one viewing orientation of a plurality of predetermined viewing orientations.

35. A method in accordance with claim 34, comprising:
selecting each predetermined viewing orientation of said plurality of viewing orientations from: a first predetermined viewing orientation looking down at a head from above, a second viewing orientation from the side looking at the side of a head, a third viewing orientation from the front looking at the front of a head, and a fourth viewing orientation from below looking up at head towards the jaw of the head.

36. In combination:
an application program operable to provide assessment of cranial deformities;
a database accessible by said application program, said database comprising a first plurality of first image sets utilizable to assess a cranial deformity type and a second plurality of second image sets utilizable to assess the severity of a cranial deformity;
said application program utilizing said database to access said first plurality of first image sets to assess a type of cranial deformity of said subject's head; and
said application utilizing said database to access said second plurality of second image sets to assess the severity of said cranial deformation of said subject's head.

37. A combination in accordance with claim 36, comprising:
said application program utilizing an assessed type of cranial deformity to select a corresponding one said second image set of said second plurality of image sets.

38. A combination in accordance with claim 36, comprising:
a set of third images of a subject's head;
said application program operable to compare said third images with said first plurality of first image sets to assess a type of cranial deformity.

39. A combination in accordance with claim 38, comprising:
said application program operable to select one of said second image sets corresponding to said assessed type of cranial deformity; and
said application program comparing said third images with said selected one of said second image sets to assess the severity of said assessed type of cranial deformity.

40. A combination in accordance with claim 36, comprising:
said first image set comprises a group of images including one image for each of a plurality of head shapes, said plurality of head shapes comprising a normal head shape and one or more head shapes selected from the group comprising a plagiocephalic head shape, a brachycephalic head shape, and a scaphocephalic head shape.

41. A combination in accordance with claim 40, comprising:
said images of said first image set are views of said corresponding head shape from one viewing orientation of a plurality of predetermined viewing orientations.

42. A combination in accordance with claim 41, comprising:
each predetermined viewing orientation of said plurality of viewing orientations selected from: a first predetermined viewing orientation looking down at a head from above, a second viewing orientation from the side looking at the side of a head, a third viewing orientation from the front looking at the front of a head, and a fourth viewing orientation from below looking up at head towards the jaw of the head.

43. A combination in accordance with claim 42, comprising:
said application program operable to utilize an assessed type of cranial deformity to select a corresponding one said second image set of said second plurality of image sets.

44. A combination in accordance with claim 42, comprising:
providing a set of third images of a subject's head;
said application program comparing said third images with said first plurality of first image sets to assess a type of cranial deformity.

45. A combination in accordance with claim 44, comprising:
said application program selecting one of said second image sets corresponding to said assessed type of cranial deformity; and
comparing said third images with said selected one of said second image sets to assess the severity of said assessed type of cranial deformity.

46. A combination in accordance with claim 36, comprising:
apparatus on which said application program is executable, said remote apparatus comprising a display.

47. A combination in accordance with claim 46, wherein:
said application program is operable on said remote apparatus to
display said selected group of images such that a user of said apparatus may compare said selected group of images with the shape of a subject's head.

48. Apparatus operable to provide assessment of cranial deformities, comprising:
programmable apparatus comprising a display and an application program
said application program including a database accessible by said application program, said database comprising a plurality of image sets;
each image set comprising a plurality of images of a corresponding head shape selected from a plurality of head shapes, each image of said plurality of images being an image of said corresponding head from one viewing orientation of a plurality of predetermined viewing orientations;
said plurality of head shapes comprising one or more head shapes selected from the group comprising a plagiocephalic head shape, a brachycephalic head shape, and a scaphocephalic head shape;
each predetermined viewing orientation of said plurality of viewing orientations being selected from a first predetermined viewing orientation looking down at a head from above, a second viewing orientation from the side looking at the side of a head, a third viewing orientation from the front looking at the front of a head, and a fourth viewing orientation from below looking up at head towards the jaw of the head; and
said application program being operable to select a group of images from said database, said selected group of images comprising a selected one image from each said image set, each said selected one image corresponding to the same one predetermined viewing orientation.

49. Apparatus in accordance with claim 48, wherein:
said application program is operable to display said selected group of images such that a user of said apparatus can compare said selected group of images with the shape of an subject's head viewed from said same one predetermined viewing orientation.

50. Apparatus in accordance with claim 48, wherein:
said application program is operable on said apparatus to select a plurality of groups of images from said database, each said selected group of images comprising a selected one image from each said image set, each said selected one image corresponding to the same one predetermined viewing orientation.

51. Apparatus in accordance with claim 50, wherein:
said application program is operable to display said plurality of groups of images on said display; and
a user of said apparatus may compare the shape of an subject's head viewed from each of said predetermined viewing orientations with a corresponding group of said plurality of groups of images.
